(19) United States
(12) Patent Application Publication
(10) Pub. No.: US 2019/0117574 A1
(43) Pub. Date: Apr. 25, 2019

(54) SURFACE-MODIFIED MICROSPHERE COMPOSITION, APPLICATION OF THE SAME, AND METHOD OF PREPARING THE SAME

(71) Applicant: INSTITUTE OF NUCLEAR ENERGY RESEARCH, ATOMIC ENERGY COUNCIL, EXECUTIVE YUAN, R.O.C., TAOYUAN CITY (TW)

(72) Inventors: YU-WEI YEN, TAINAN CITY (TW); TSAI-YUEH LUO, TAOYUAN CITY (TW); CHENG-LIANG PENG, TAIPEI CITY (TW); PING-FANG CHIANG, TAOYUAN CITY (TW)

(21) Appl. No.: 15/788,955

(22) Filed: Oct. 20, 2017

(51) Int. Cl. A61K 9/16; A61K 31/196

(52) U.S. Cl. CPC A61K 9/167; A61K 9/1647; A61K 9/1635; A61K 31/196; A61K 9/1652

(57) ABSTRACT

A surface-modified microsphere composition comprises poly(lactic-co-glycolic acid) (PLGA), chitosan, hydrophobic drug, and hydrophilic drug. The PLGA forms a microsphere. The chitosan is formed on a surface of the microsphere. The hydrophobic drug is encapsulated by the microsphere. The hydrophilic drug is adsorbed on the surface of the microsphere. The surface-modified microsphere composition is for treating arthritis. A method of preparing a surface-modified microsphere composition involves producing by an oil-in-water emulsion method microspheres formed from PLGA, covered with chitosan, and adapted to encapsulate hydrophobic drug, and having hydrophilic drug adsorbed on their surfaces. The surface-modified microsphere composition enables drug to be released to achieve efficacy both instantly and persistently.

### FIELD OF THE INVENTION

The present invention relates to surface-modified microsphere compositions, applications of the same, and methods of preparing the same and, more particularly, to a surface-modified microsphere composition, an application of the same, and a method of preparing the same, characterized in that the surface-modified microsphere composition thus prepared is for use in dual-stage drug release.

### BACKGROUND OF THE INVENTION

In drug delivery systems (DDS), microspheres are widely used to control drug release. In this regard, microspheres are microscopic spheres in a range of 0.1-200 μm, which are made of biodegradable materials or non-biodegradable materials and applied to delivery of hydrophobic or hydrophilic drugs as well as serving as vehicles for bioactive molecules. Take biodegradable microspheres as an example, the drug molecules release initially occurs by diffusion through the matrix or the pores of microspheres, and then the drug is released as a result of decomposition of the microspheres. According to the prior art, hydrophilic drugs encapsulated by hydrophobic polymer poly(lactic-co-glycolic acid) (PLGA) microspheres, are released over a short period of time, whereas hydrophobic drugs encapsulated by PLGA microspheres are released persistently over a long period of time. From a therapeutic perspective, hydrophilic drugs encapsulated microspheres can act quickly but fail to serve as a long-acting topical medication, whereas hydrophobic drugs encapsulated microspheres can serve as a long-acting topical medication but are limitedly effective after initial dosage because of a slow decomposition of the microspheres. As a result, conventional microspheres fail to achieve effective drug levels rapidly after initial dosage and to keep releasing drugs persistently at the same time.

### SUMMARY OF THE INVENTION

It is an objective of the present invention to provide a surface-modified microsphere composition which comprises: poly(lactic-co-glycolic acid) (PLGA) for forming a microsphere; chitosan formed on the surface of the microsphere; hydrophobic drug encapsulated by the microsphere; and hydrophilic drug adsorbed on the surface of the microsphere.

Regarding the surface-modified microsphere composition, the hydrophobic drug comprises one selected from the group consisting of diclofenac, ibuprofen, celecoxib, indomethacin, and dexamethasone.

Regarding the surface-modified microsphere composition, the hydrophilic drug is diclofenac sodium or dexamethasone sodium phosphate.

Regarding the surface-modified microsphere composition, the surface of the microsphere carries positive charges.

The surface-modified microsphere composition further comprises 50-90% w/v PLGA, 2-10% w/v chitosan, 10-50% w/v diclofenac free acid, and 2-10% w/v diclofenac sodium.

In order to achieve the above and other objectives, the present invention provides an application of the surface-modified microsphere composition, wherein the surface-modified microsphere composition is for treating arthritis.

In order to achieve the above and other objectives, the present invention provides a method of preparing a surface-modified microsphere composition, comprising the steps of: (a) dissolving poly(lactic-co-glycolic acid) (PLGA) and hydrophobic drug in ethyl acetate solution; (b) introducing an aqueous solution inclusive of chitosan and an emulsifying agent into the ethyl acetate solution and homogenizing the mixture to form an emulsion containing microspheres; (c) allowing the emulsion to undergo rotary evaporation, thereby producing a suspension containing the microspheres; (d) removing water from the suspension to obtain the microspheres; (e) introducing the microspheres into a buffer solution inclusive of hydrophilic drug while stirring the buffer solution; and (f removing water from the buffer solution to obtain the surface-modified microsphere composition.

Regarding the method, the hydrophobic drug comprises one selected from the group consisting of diclofenac, ibuprofen, celecoxib, indomethacin and dexamethasone.

Regarding the method, the hydrophilic drug is diclofenac sodium or dexamethasone sodium phosphate.

Regarding the method, surfaces of the microspheres carry positive charges.

Regarding the method, the emulsifying agent is polyvinyl alcohol (PVA).

The surface-modified microsphere composition and the method of preparing the same enable drug to be released to achieve efficacy both instantly and persistently.

### BRIEF DESCRIPTION OF THE DRAWINGS

Objectives, features, and advantages of the present invention are hereunder illustrated with specific embodiments in conjunction with the accompanying drawings, in which:

FIG. 1 is a schematic view of releasing drug with a surface-modified microsphere according to an embodiment of the present invention;

FIG. 2 is a graph of distribution of sizes of surface-modified microspheres formed from the surface-modified microsphere composition according to an embodiment of the present invention;

FIG. 3 is a graph of comparison in distribution of zeta potential between conventional PLGA microspheres and the surface-modified microspheres formed from the surface-modified microsphere composition according to an embodiment of the present invention;

FIG. 4 are thermal images taken of a mouse on day 0, day 3, and day 14 after injected with FCA, respectively, according to an embodiment of the present invention;

FIG. 5 is an IVIS image taken of FCA-induced arthritis mice on day 21 after administered with a neutrophil elastase 680 FAST probe according to an embodiment of the present invention;

FIG. 6 are pictures taken of mice on day 21 after injected with FCA according to an embodiment of the present invention;

FIG. 7 is a graph of diameters of ankle joints of left and right hind legs of mice with FCA-induced arthritis against the number of days after injected with FCA according to an embodiment of the present invention;

FIG. 8 are graphs of diameters of ankle joints of left and right hind legs of mice with FCA-induced arthritis against the number of days after treatment according to an embodiment of the present invention;

FIG. 9 is a bar chart of temperature of ankle joints of right hind legs of mice with FCA-induced arthritis against the number of days after treatment according to an embodiment of the present invention; and

FIG. 10 are graphs of body weight of mice with FCA-induced arthritis against the number of days after treatment according to an embodiment of the present invention.

### DETAILED DESCRIPTION OF THE PREFERRED EMBODIMENTS

A method of preparing a surface-modified microsphere composition according to an embodiment of the present invention is described below.

In this embodiment, the surface-modified microsphere composition is prepared by an oil-in-water (o/w) emulsion method.

Poly(lactic-co-glycolic acid) (PLGA) (5% w/v) and diclofenac free acid (DFA) (0.5-5% w/v) are dissolved in an ethyl acetate solution, wherein DFA is a hydrophobic drug. The mixture of the PLGA, DFA and ethyl acetate is introduced drop by drop into a 40 ml of aqueous solution which contains polyvinyl alcohol (PVA) (1% w/v) and chitosan (0.3% w/v) and then processed with a homogenizer for 10 minutes to undergo emulsification. PVA functions as an emulsifying agent and a stabilizing agent, but the present invention is not limited thereto. Owing to the emulsification, the aforesaid solution turns into an emulsion containing microspheres. The microspheres are each mainly formed from PLGA and covered with chitosan. Chitosan serves to modify surfaces of the microspheres so that the surfaces of the surface-modified microspheres carry positive charges. DFA is encapsulated by the microspheres.

The emulsion undergoes evaporation in a rotary evaporator for 30 minutes to remove the organic solvent from the emulsion. Upon completion of the rotary evaporation process, the emulsion produces a suspension. The suspension contains the microspheres. The microspheres in the suspension are rinsed by distilled water thrice to further remove residual molecules from the surfaces of the microspheres. Afterward, the suspension undergoes centrifugation and freeze-drying overnight to obtain dried microspheres.

The dried microspheres are dissolved, to attain a concentration of 1.5% w/v, in phosphate buffered saline (PBS) solution containing diclofenac sodium (1.5% w/v).

The PBS solution, which contains microspheres (1.5% w/v) and diclofenac sodium (1.5% w/v), is shaken at room temperature for 30 minutes so that the negative-charged hydrophilic drug (diclofenac sodium) is adsorbed on the positive-charged surfaces of the microspheres to form a surface-modified microsphere composition. Free diclofenac sodium (free DS) is removed from the aforesaid PBS solution-containing suspension, which contains microspheres (1.5% w/v) and diclofenac sodium (1.5% w/v), by rinsing the aforesaid suspension with distilled water and processing the rinsed PBS solution, thrice. Afterward, the PBS solution, which the surface-modified microsphere composition is dissolved in, is freeze-dried overnight to remove water and thus obtain the surface-modified microsphere composition. Finally, the surface-modified microsphere composition is stored at 4° C.

Another embodiment of the method of preparing the surface-modified microsphere composition features PLGA concentration of 1-10% w/v, DFA concentration of 0.1-5% w/v, PVA concentration of 0.5-2% w/v, chitosan concentration of 0.1-1% w/v, and microspheres and diclofenac sodium concentration of 0.5-5% w/v (in the PBS solution.) The concentrations of respective ingredients for use with the method of preparing the surface-modified microsphere composition depend on the preparation process and thus are subject to changes, thereby not being restricted to this embodiment.

In an embodiment, the surface-modified microsphere composition essentially comprises PLGA, chitosan, hydrophobic drug, and hydrophilic drug. The PLGA is for forming a microsphere. The chitosan is formed on the surface of the microsphere. The hydrophobic drug is encapsulated by the microsphere. The hydrophilic drug is adsorbed on the surface of the microsphere. The hydrophobic drug delivered is DFA, whereas the hydrophilic drug delivered is diclofenac sodium. However, in a variant embodiment, in the process of preparing the surface-modified microsphere composition, any other hydrophobic drug and hydrophilic drug substitute for DFA and diclofenac sodium, respectively, so that the surface-modified microsphere composition is conducive to delivery of the other types of drug. For instance, the hydrophobic drug encapsulated by the microsphere is diclofenac, ibuprofen, celecoxib, indomethacin, and dexamethasone, whereas the hydrophilic drug adsorbed on the surface of the microsphere is diclofenac sodium or dexamethasone sodium phosphate, thereby not being restricted to this embodiment. In this embodiment, the surface-modified microsphere composition comprises 86% w/v PLGA, 2% w/v chitosan, 10% w/v DFA, and 2% w/v diclofenac sodium. In another embodiment, the surface-modified microsphere composition comprises 50-90% w/v PLGA, 2-10% w/v chitosan, 10-50% w/v DFA, and 2-10% w/v diclofenac sodium, and the concentrations of the respective ingredients of the surface-modified microsphere composition are subject to changes as needed, thereby not being restricted to this embodiment.

Analysis of Zeta Potential and Size Distribution of Surface-modified Microspheres Formed from Surface-modified Microsphere Composition

The size distribution of surface-modified microspheres formed from the surface-modified microsphere composition is analyzed with Sysmex FPIA 300, which is manufactured by Malvern Instruments Ltd., the UK.

Both non-modified microspheres and surface-modified microsphere composition dissolve in the PBS solution which has a pH of 7.4, and then the zeta potential of non-modified microspheres and surface-modified microsphere composition is measured with Zetasizer Nano Z, which is manufactured by Malvern Instruments Ltd., the UK.

Animal Model Created with Arthritis Induced by Freund's Complete Adjuvant (FCA) 24 male mice (C57BL/6, purchased from the National Laboratory Animal Center, Taipei, Taiwan) weighing 20-25 g (8 weeks old) are provided. The mice are each given a subdermal injection of 0.1 ml of FCA at the tibiotarsal joint of the right hind leg and 0.05 ml of FCA on the subplantar side of the right hind paw to induce the development of arthritis. Afterward, thermal images are taken of the mice with a thermal imaging camera on day 0, day 3, and day 14 after injected with FCA, respectively. The diameters of ankle joints of left and right hind legs of the mice are measured with a vernier caliper are surveyed and recorded for 2 or 3 days. The mice with FCA-induced arthritis are administered with a neutrophil elastase 680 FAST probe through trail veins on day 21 and observed with IVIS (non-invasion in vivo imaging system) to confirm whether neutrophil elastase is produced at the location where FCA is injected.

Test of Treating Mice with FCA-induced Arthritis

The animal model created with FCA-induced arthritis is described above. 24 mice which conform to the animal model created with FCA-induced arthritis are provided. Experiments of therapeutic assessment are conducted, with the surface-modified microsphere composition, on the mice on day 21 after injected with FCA. The 24 mice are randomly divided into four groups (that is, six mice per group.) The four groups of mice are administered with reagents, namely saline, free diclofenac sodium, slow release PLGA microsphere, and the surface-modified microsphere composition, respectively. The mice are administered with 10 mg/kg/ml of diclofenac sodium (dissolved in the saline). The slow release PLGA MS contains DFA. The mice administered with the slow release PLGA MS end up with 10 mg/kg/ml of DFA. The surface-modified microsphere composition comprises diclofenac sodium and DFA; hence, the mice administered with the surface-modified microsphere composition end up with 10 mg/kg/ml of diclofenac sodium and DFA. All the aforesaid reagents are injected subdermally around the tibiotarsal joints of the mice's right hind legs.

Upon completion of the subdermal injection of the reagents into the four groups of mice, the diameters of left and right ankle joints and body weight of the mice are measured with a vernier caliper and recorded for 2 or 3 days.

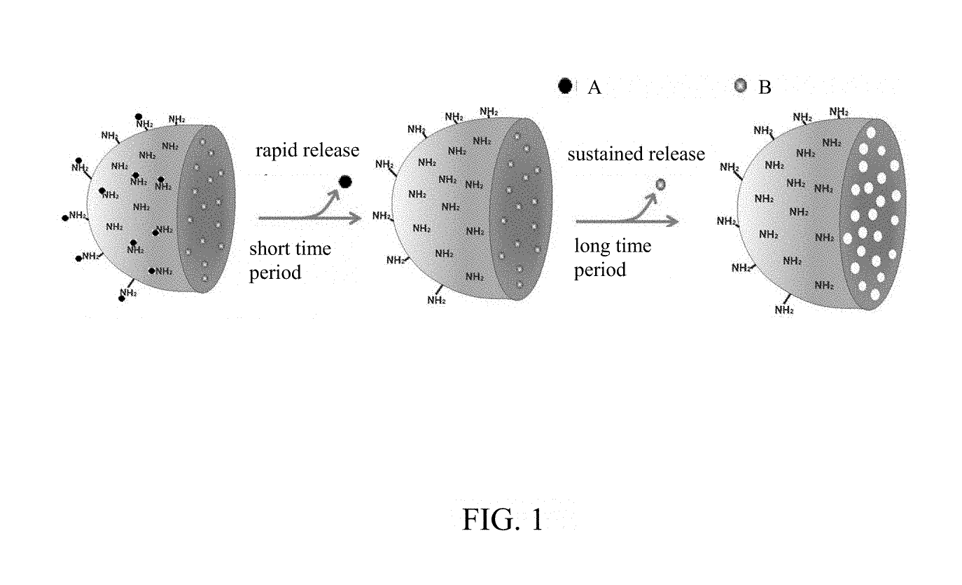

FIG. 1 is a schematic view of releasing drug with a surface-modified microsphere according to this embodiment. The modified spherical surfaces of the microspheres formed from the surface-modified microsphere composition carry positive charges; hence, drug is carried in both the inside of the microspheres and spherical surfaces thereof to effectuate dual-stage drug release: quick release, over a short period of time, of hydrophilic diclofenac sodium A otherwise adsorbed on the surfaces of the microspheres, followed by persistent release, over a long period of time, of DFA B otherwise encapsulated by the microspheres. The dual-stage drug release, coupled with the surface-modified microsphere composition, enables drug to be released to achieve efficacy both instantly and persistently.

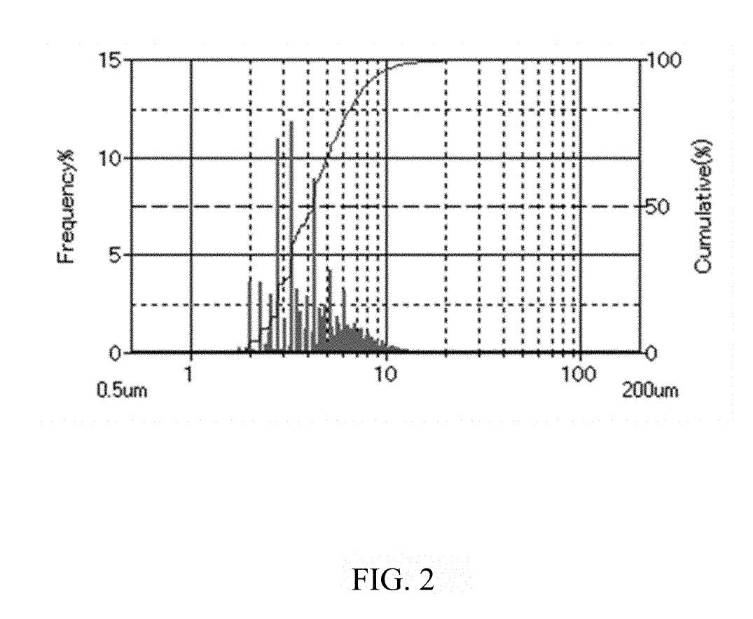

FIG. 2 is a graph of distribution of sizes of surface-modified microspheres formed from the surface-modified microsphere composition according to this embodiment, showing that the surface-modified microspheres formed from the surface-modified microsphere composition are around 1-10 μm in diameter.

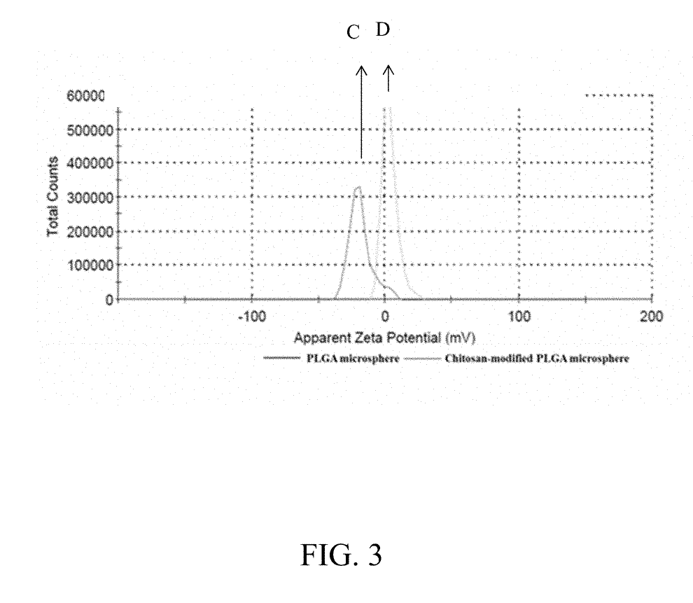

FIG. 3 is a graph of comparison in distribution of zeta potential between conventional PLGA microspheres C and the surface-modified microspheres formed from the surface-modified microsphere composition D in this embodiment. As shown in the graph, unlike the conventional PLGA microspheres C, the surface-modified microspheres have their zeta potential falling within the range of positive charges, indicating that the surfaces of the surface-modified microspheres carry positive charges.

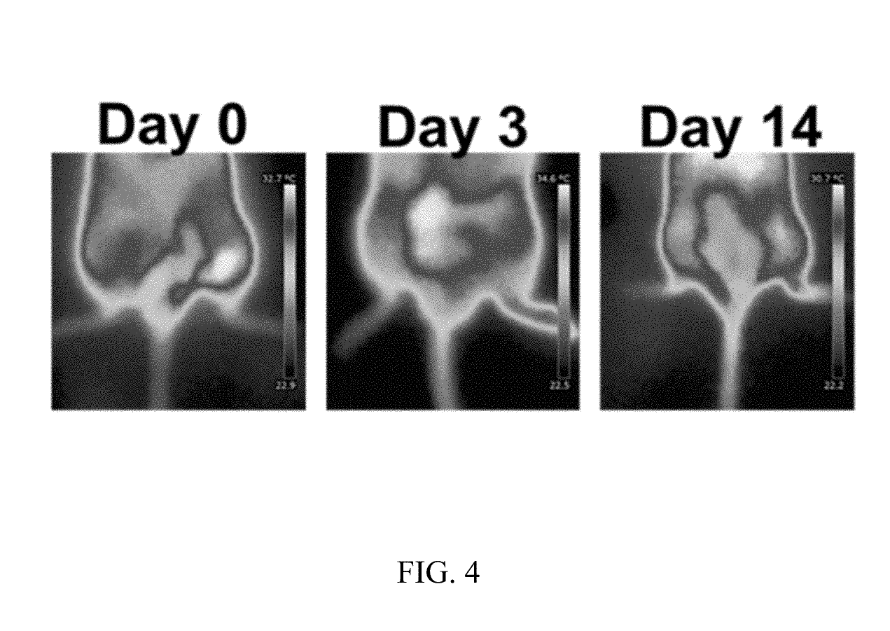

FIG. 4 are thermal images taken of a mouse on day 0, day 3, and day 14 after injected with FCA, respectively. The thermal images show that the right hind leg of the mouse injected with FCA has obviously higher temperature and has developed inflammation.

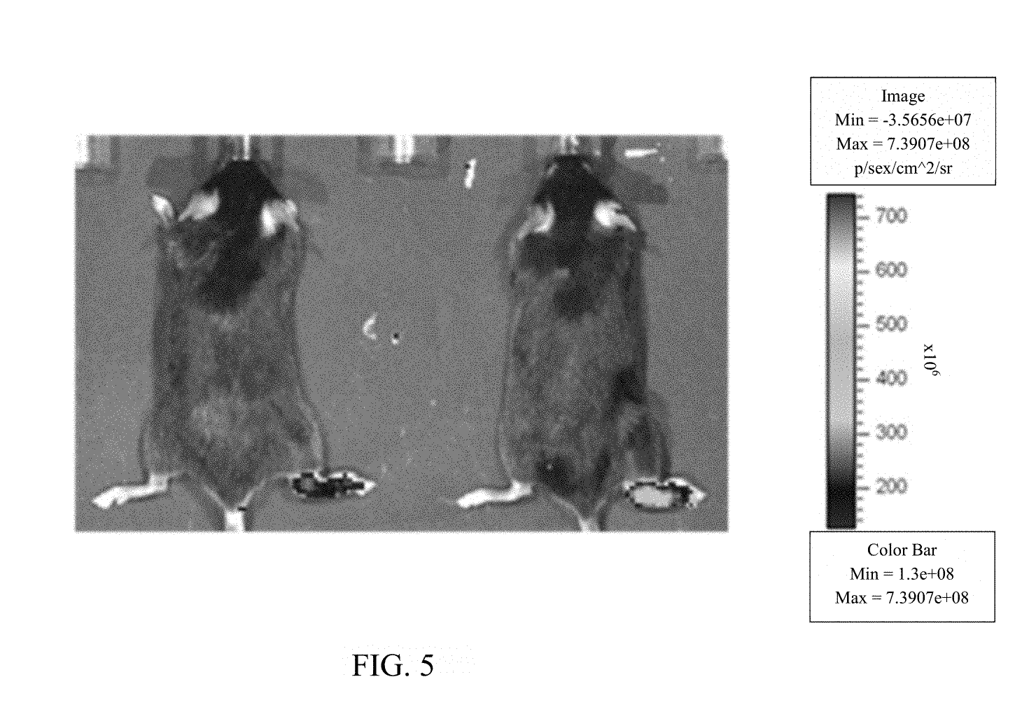

FIG. 5 is an IVIS image taken of FCA-induced arthritis mice on day 21 after administered with a neutrophil elastase 680 FAST probe. The IVIS image shows that neutrophil elastase is secreted and accumulated at the right ankles of the mice injected with FCA. Neutrophil elastase is secreted during inflammation; hence, the detection of presence of neutrophil elastase at the right ankles of the mice with FCA-induced arthritis indicates that the mice's right ankles have developed inflammation.

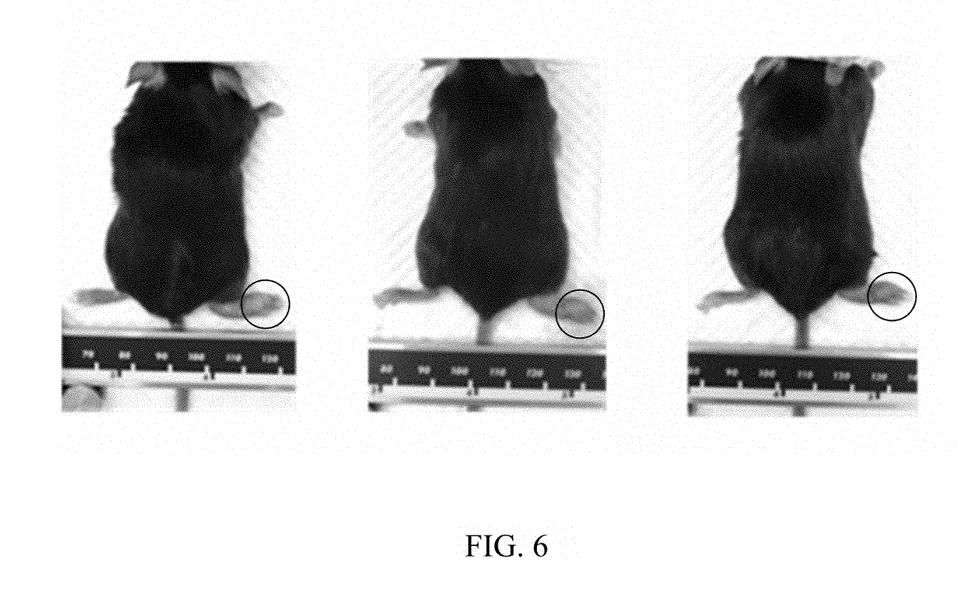

FIG. 6 are pictures taken of mice on day 21 after injected with FCA. The pictures show that, compared with the left ankles, the right ankles of the mice with FCA-induced arthritis are markedly swollen.

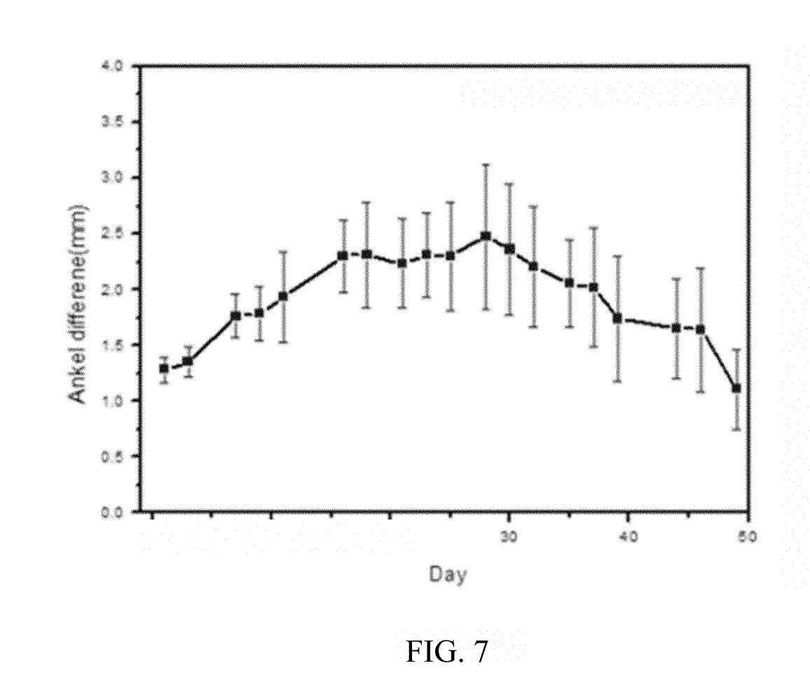

FIG. 7 is a graph of diameters of ankle joints of left and right hind legs of mice with FCA-induced arthritis against the number of days after injected with FCA. The graph shows that, after being injected with FCA, the mice have their right ankles swollen gradually because of inflammation, and the swelling of the right ankles begin to abate on day 30 after injected with FCA.

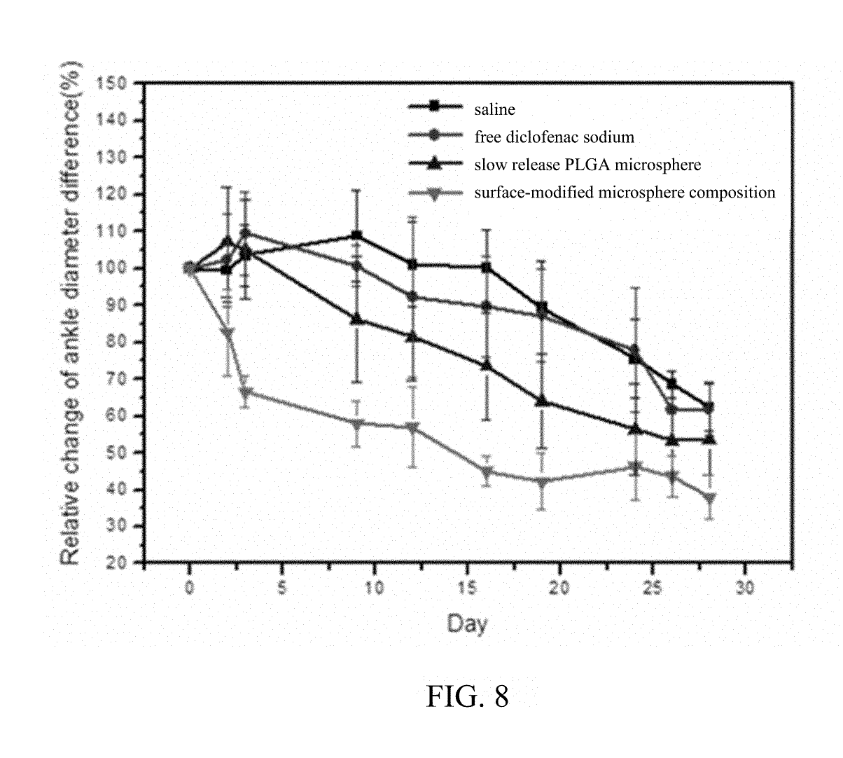

FIG. 8 are graphs of diameters of ankle joints of left and right hind legs of mice with FCA-induced arthritis against the number of days after treatment. The graphs show the course of the swelling of the mice's right ankles and the reduction thereof. The graphs not only show that free diclofenac sodium, slow release PLGA MS, and the surface-modified microsphere composition are more effective than saline in reducing the swelling of the mice's ankles, but also show that the surface-modified microsphere composition surpasses free diclofenac sodium and slow release PLGA MS in reducing the swelling of the mice's ankles.

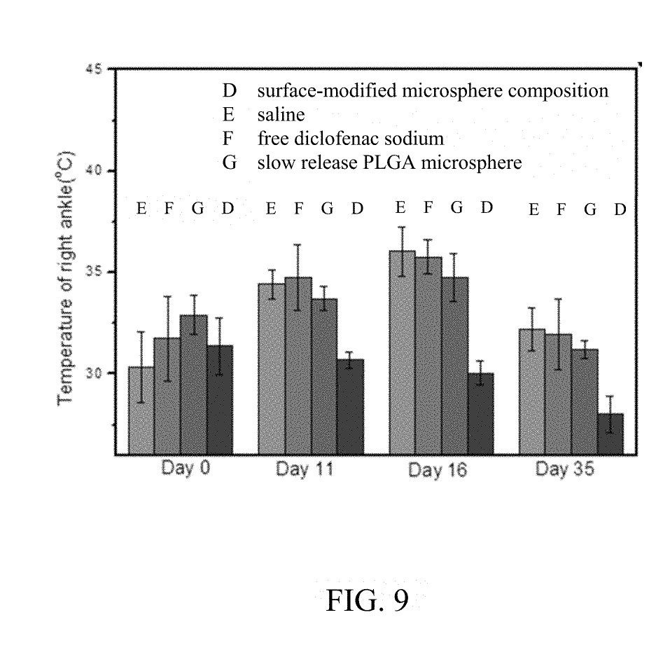

FIG. 9 is a bar chart of temperature of ankle joints of right hind legs of mice with FCA-induced arthritis against the number of days after treatment. The bar chart shows that right ankles of the mice administered with the surface-modified microsphere composition D have markedly lower temperature than right ankles of the mice administered with saline E, free diclofenac sodium F and slow release PLGA MS G on whichever day, and that the temperature of the right ankles of the mice administered with the surface-modified microsphere composition D drops gradually but never rises.

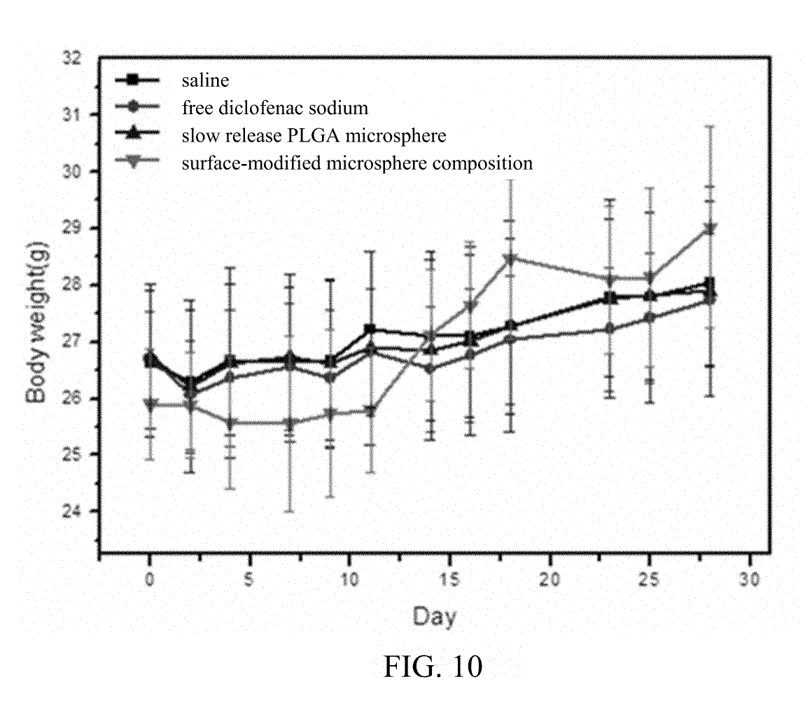

FIG. 10 are graphs of body weight of mice with FCA-induced arthritis against the number of days after treatment. The graphs show that on day 30 after treatment, compared with the mice administered with saline, free diclofenac sodium and slow release PLGA MS, the mice administered with the surface-modified microsphere composition gain weight markedly rather than lose weight which is otherwise caused by negative side effects.

In this embodiment, the surface-modified microsphere composition is for use in treating arthritis. However, in a variant embodiment, the surface-modified microsphere composition carries the other types of drug to treat the other types of diseases, such as cancer or eye diseases.

Surface-modified microspheres formed from the surface-modified microsphere composition of the present invention have spherical surfaces which are modified to carry positive charges so that drug is carried inside the surface-modified microspheres and the spherical surfaces of the surface-modified microspheres to effectuate dual-stage drug release. Initially, the surface-modified microspheres quickly release hydrophilic drug otherwise adsorbed on the spherical surfaces of the surface-modified microspheres. Afterward, the surface-modified microspheres persistently release hydrophobic drug from the inside of the surface-modified microspheres. Therefore, the surface-modified microsphere composition of the present invention enables drug to be released to achieve efficacy both instantly and persistently.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

1. A surface-modified microsphere composition, comprising:
a microsphere made of poly(lactic-co-glycolic acid) (PLGA);
chitosan covered on a surface of the microsphere;
hydrophobic drug encapsulated by the microsphere; and
hydrophilic drug adsorbed on the chitosan-covering surface of the microsphere.

2. The surface-modified microsphere composition of claim 1, wherein the hydrophobic drug comprises one selected from the group consisting of diclofenac, ibuprofen, celecoxib, indomethacin, and dexamethasone.

3. The surface-modified microsphere composition of claim 1, wherein the hydrophilic drug is one of diclofenac sodium and dexamethasone sodium phosphate.

4. The surface-modified microsphere composition of claim 1, comprising 50-90% w/w of PLGA, 2-10% w/w of chitosan, 10-50% w/w of diclofenac free acid, and 2-10% w/w of diclofenac sodium, based on a total weight of the surface-modified microsphere composition.

5. An application of the surface-modified microsphere composition of claim 1, wherein the surface-modified microsphere composition of claim 1 is for treating arthritis.

6. An application of the surface-modified microsphere composition of claim 2, wherein the surface-modified microsphere composition of claim 2 is for treating arthritis.

7. An application of the surface-modified microsphere composition of claim 3, wherein the surface-modified microsphere composition of claim 3 is for treating arthritis.

8. An application of the surface-modified microsphere composition of claim 4, wherein the surface-modified microsphere composition of claim 4 is for treating arthritis.

9. A method of preparing a surface-modified microsphere composition, comprising the steps of:
(a) dissolving poly(lactic-co-glycolic acid) (PLGA) and hydrophobic drug in ethyl acetate solution;
(b) introducing an aqueous solution inclusive of chitosan and an emulsifying agent into the ethyl acetate solution and homogenizing the mixture to form an emulsion containing microspheres;
(c) allowing the emulsion to undergo rotary evaporation, thereby producing a suspension containing the microspheres;
(d) removing water from the suspension to obtain the microspheres;
(e) introducing the microspheres into a buffer solution inclusive of hydrophilic drug while stirring the buffer solution; and
(f) removing water from the buffer solution to obtain the surface-modified microsphere composition.

10. The method of claim 9, wherein the hydrophobic drug comprises one selected from the group consisting of diclofenac, ibuprofen, celecoxib, indomethacin, and dexamethasone.

11. The method of claim 9, wherein the hydrophilic drug is one of diclofenac sodium and dexamethasone sodium phosphate.

12. The method of claim 9, wherein surfaces of the microspheres carry positive charges.

13. The method of claim 9, wherein the emulsifying agent is polyvinyl alcohol (PVA).
